(19) United States
(12) Patent Application Publication
(10) Pub. No.: US 2018/0317875 A1
(43) Pub. Date: Nov. 8, 2018

(54) System and Method for Telemetrically Monitoring a Target Object

(71) Applicants: Andrey Bakhriddinovich Khayrullaev, Pague (CZ); Evgeniy Vacheslavovich Borodin, Samara (RU); Maxim Anatoljevich Maltsev, Prague (CZ)

(72) Inventors: Andrey Bakhriddinovich Khayrullaev, Pague (CZ); Evgeniy Vacheslavovich Borodin, Samara (RU); Maxim Anatoljevich Maltsev, Prague (CZ)

(21) Appl. No.: 15/586,197

(22) Filed: May 3, 2017

(51) Int. Cl. A61B 7/04; A61B 5/22; A61B 5/00

(52) U.S. Cl. CPC A61B 7/04; A61B 5/0245; A61B 5/6824; A61B 5/222

(57) ABSTRACT

A wearable telemetry device for monitoring a target object, for example, a baby, includes a strap and an enclosure that encloses components including a processor, sensors, an audio capture device, an output device, and a rechargeable power unit. The processor executes modules of the wearable telemetry device for receiving user input data from a user device, facilitating communication between the target object and the user device via a transceiver, activating and controlling the sensors, the audio capture device, and the output device based on the user input data, filtering sensor data, computing monitoring indexes by processing health parameters, motion, and/or position of the target object detected by the sensors, in accordance with threshold data, ambient audio signals received by the audio capture device, and/or the user input data, and generating and transmitting alert notifications based on the monitoring indexes to the target object, and/or the user device via the transceiver.

### BACKGROUND OF THE INVENTION

### Field of the Invention

The system and method disclosed herein, in general, relate to telemetry. More particularly, the system and method disclosed herein relate to a wearable telemetry device, and more specifically, to a system and method for monitoring a target object, for example, a baby.

### Description of the Related Art

Systems and methods for telemetrically monitoring target objects, such as adult patients, children, babies, or infants are known in the art.

For example, U.S. Pat. No. 7,364,539 issued to M. Mackin on Apr. 29, 2008 discloses an infant warming apparatus for supporting an infant upon an infant bed. The apparatus has a sensor that is affixed to the skin of the infant to detect one or more physiological functions of the infant. A transmitter is located within the enclosure of the sensor which transmits the information detected by the physiological sensor to a receiver that is located on the infant care apparatus and which can then convert that information into a recognizable or usable medium. An alternative embodiment includes the transmitter located proximate to the infant within an infant scale located beneath the infant. The sensor is hardwired to the transmitter in the infant scale and signals relating to weight and/or a condition of the infant are transmitted by wireless telemetry to a monitor or other display device to display that information to the caregiver.

U.S. Pat. No. 9,028,405 issued on May 12, 2015 to Bao Tran discloses a system that includes a processor; a cellular Wi-Fi, or Bluetooth® transceiver coupled to the processor; an accelerometer or a motion sensor coupled to the processor; and a sensor coupled to the processor to sense mood, wherein text, image, sound, or video is rendered in response to the sensed mood. Among other parameters, the system controls and monitors a heart rate, a blood pressure, heart beat sounds, bio-electric impedance, etc. The system may also include an accelerometer to detect a dangerous condition such as a falling condition and to generate a warning when the dangerous condition is detected. An electrode or electrodes of the sensor mounted on the mobile telephone case may be used to contact the person's skin and capture bio-electrical signals, and the amplifier coupled to the electrodes, a processor coupled to the amplifier, and a screen coupled to the processor allow to display medical data such as images of the bio-electrical signals.

U.S. Pat. No. 5,652,570 issued on Jul. 29, 1997 to R. Lepkofker discloses an interactive individual location and monitoring system includes a central monitoring system for maintaining health, location, and other data with respect to an individual. A watch unit carried by the individual receives medical and other information selected by and inputted directly from the individual. The watch unit broadcasts the medical and other information locally by radio in a region near the individual. Preferably, the present invention includes an embodiment which also monitors vital signs of a user. The pod unit includes a triaxial accelerometer for gathering acceleration data for transmission of the data to the central monitoring station for analysis at a later time. The central monitoring system broadcasts alerts and queries directed to the individual and the transponder pod unit receives and rebroadcasts the alerts and queries locally. The watch unit receives the alerts and queries, and the watch unit includes a vibratory annunciator which alerts the individual of an inquiry signal from the pod unit.

German Patent No DE 10352591 issued on Jun. 16, 2005 to Anja Falk, et al. discloses a device for monitoring vital parameters, especially of baby. The device, has wireless data transmission module; sensors, signal processing unit; transmission module and power supply unit which are integrated into a watch in miniaturized form. The device incorporates measurement of vital signs.

Although the list of such examples may be continued with reference to many other similar devices and methods, there is still enough room for improvement since in many cases such devices do not take into account automatic corrections of wrong data and do not convert data between various conversion protocols. Also, in a majority of cases, monitoring devices for individuals relate to adult patients or to infants whose motions are restricted by a bed or fencing. However, a baby who is about two years old is normally very mobile and requires extremely cautious watching. The baby's skin is thin, and this allows to obtain accurate measurement of vital parameters through skin contact. Nevertheless, the inventions relating to telemetric monitoring of children in this category are few in number and are still on a demand.

### SUMMARY OF THE INVENTION

Disclosed herein is a wearable telemetry device and method for monitoring a target object, for example, a baby. The wearable telemetry device comprises a strap and an enclosure. The strap is wearable by the target object. The strap comprises a cavity positioned at a preconfigured location, for example, a central location of the strap. The enclosure is detachably positioned within the cavity of the strap. The enclosure encloses multiple telemetry device components therewith in. The telemetry device components comprise at least one processor, multiple sensors, an audio capture device, a non-transitory computer readable storage medium, an output device, and a rechargeable power unit. The sensors and the audio capture device are communicatively coupled to the processor. The sensors generate sensor data by detecting health parameters, and/or motion, and/or position of the target object. The audio capture device receives ambient audio signals. The non-transitory computer readable storage medium stores the generated sensor data, the received ambient audio signals, threshold data, preconfigured media data, and instructions defined by modules of the wearable telemetry device.

The processor of the wearable telemetry device is communicatively coupled to the non-transitory computer readable storage medium and operably coupled to a transceiver. The processor executes one or more computer program instructions defined by the modules of the wearable telemetry device. The modules of the wearable telemetry device comprise data communication modules, control modules, data processing modules, and an alerting module. The data communication module receives user input data from a monitoring application deployed on one or more user devices via the transceiver and facilitates communication between the target object and the user devices via the transceiver. The control module activates and controls operability and sensitivity of the sensors, and/or the audio capture device, and/or the output device based on the received user input data. The data processing module filters the sensor data generated by the activated sensors and computes monitoring indexes by processing the detected health parameters, and/or the motion, and/or the position of the target object from the filtered data in accordance with the threshold data, and/or the received ambient audio signals, and/or the received user input data. The alerting module generates alert notifications in multiple formats based on the computed monitoring indexes and transmits the generated alert notifications to the target object and/or the user devices via the transceiver. The output device is communicatively coupled to the processor and the transceiver via a codec. The output device plays the preconfigured media data and/or the data received from the processor and/or the transceiver via the codec. The rechargeable power unit is operably coupled to the processor, the transceiver, the sensors, the audio capture device, and the output device for powering the processor, the transceiver, the sensors, the audio capture device, and the output device.

### BRIEF DESCRIPTION OF THE DRAWINGS

FIG. 1A exemplarily illustrate perspective views of a wearable telemetry device for monitoring a target object.

FIG. 1B exemplarily illustrates a bottom view of the wearable telemetry device for monitoring a target object.

FIG. 2 exemplarily illustrates a perspective view of the wearable telemetry device positioned on an arm of a target object.

FIG. 3 exemplarily illustrates a perspective view of the wearable telemetry device positioned on a wrist of a target object.

FIG. 4 exemplarily illustrates a block diagram showing telemetry device components of the wearable telemetry device.

FIG. 5 exemplarily illustrates wireless communication between the wearable telemetry device and a base station.

FIG. 6 exemplarily illustrates a system for monitoring a target object, showing wireless communication between the wearable telemetry device, the base station, and multiple user devices.

FIG. 7 exemplarily illustrates a modification of the system for monitoring a target object, showing wireless communication between the wearable telemetry device and multiple user devices.

FIGS. 8A-8B exemplarily illustrate configuration of dangerous spots in a region using a monitoring application deployed on a user device for monitoring a target object.

FIG. 9 exemplarily illustrates monitoring of a target object in an environment with dangerous spots identified using a monitoring application deployed on a user device.

FIG. 10 exemplarily illustrates monitoring of a target object in an environment with a safe area identified using a monitoring application deployed on a user device.

FIG. 11 exemplarily illustrates a flowchart comprising the steps of determining a heart rate of a target object using a heart-rate output data determination and a correlation block of the wearable telemetry device.

FIG. 12 exemplarily illustrates a flowchart comprising the steps of determining an oxygen saturation level in the blood of a target object using an output SPO2 value determination and correlation block of the wearable telemetry device.

FIG. 13 exemplarily illustrates a flowchart comprising the steps for determining a reference point to set a temperature extreme of thermal comfort of a target object using the wearable telemetry device worn by the target object.

FIG. 14 exemplarily illustrates a flowchart comprising the steps for reproducing sound of a target object on a user device using the wearable telemetry device worn by the target object.

FIG. 15 exemplarily illustrates a flowchart comprising the steps for initiating wireless transmission of sensor data to a user device or the base station proximal to the wearable telemetry device via a wireless communication protocol based on a deviation found in the sensor data.

FIG. 16 exemplarily illustrates a flowchart comprising the steps for transmitting alert notifications to a user device or the base station on determining a deviation of measured data from threshold data.

FIG. 17 exemplarily illustrates confirmation of task accomplishment by a user by tapping on the enclosure of the wearable telemetry device.

FIG. 18 exemplarily illustrates a screenshot that is provided by the monitoring application deployed on the user device for setting up tasks and tracking task accomplishment and that graphically represents user's experience.

### DETAILED DESCRIPTION

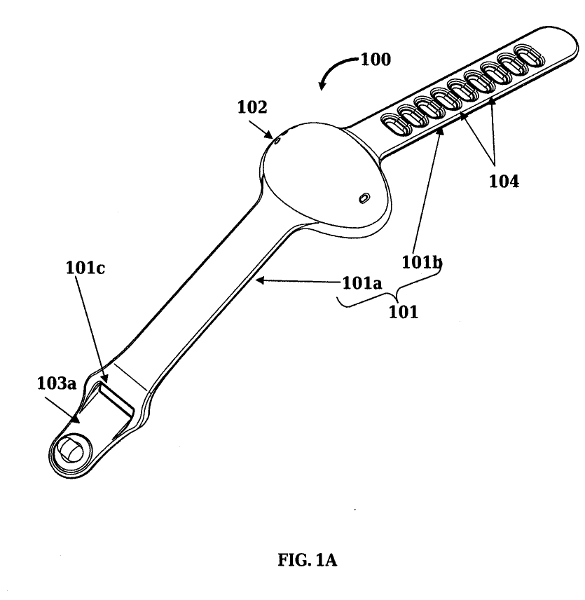

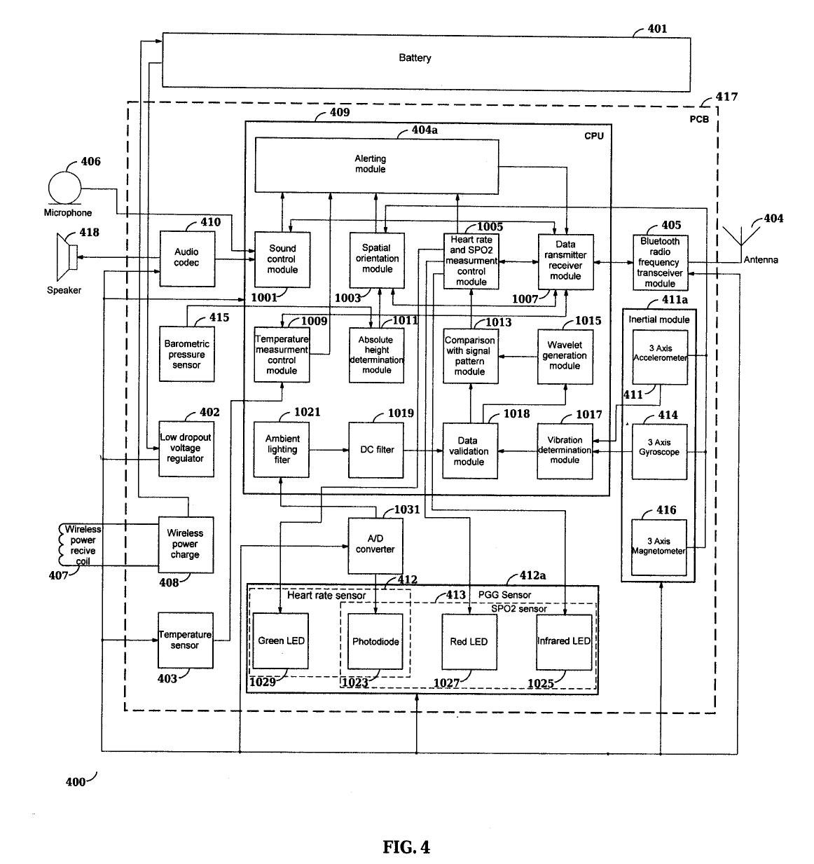

FIG. 1A exemplarily illustrates a perspective view of a wearable telemetry device 100 of the invention for monitoring a target object. As used herein, the term “target object” refers to an entity, for example, a child, e.g., a baby up to two years of age who is monitored using the wearable telemetry device 100. The wearable telemetry device may have, e.g., the following dimensions: 13 mm×30 mm×22 mm. The wearable telemetry device 100 comprises a strap 101 and an enclosure 102. The strap 101 is wearable by the target object, for example, on a wrist or an arm. The strap 101 comprises two parts 101a and 101b. The enclosure 102 encloses multiple telemetry device components disclosed in the detailed description below and shown schematically in FIG. 4. The strap 101 may be embedded into the enclosure forming with the wearable telemetry device 100 an integral non-divisible structure. The wearable telemetry device 100 can be securely fastened on an arm or a wrist of the target object. The wearable telemetry device 100 further comprises a clasp 103a positioned on one end 101c of the part 101a of the strap 101 and openings 104 configured on the part 101b of the strap 101. The clasp 103a on the part 101a of the strap 101 is constructed to engage and lock the openings 104 on the part 101b of the strap 101 to prevent, for example, a baby from unlocking the wearable telemetry device 100 without parental supervision. The wearable telemetry device 100 disclosed herein can be used indoors and outdoors.

FIG. 1B exemplarily illustrates a bottom view of the wearable telemetry device 100 for monitoring parameters of the target object, for example, a baby. A bottom portion 102a of the enclosure 102 of the wearable telemetry device 100 is exposed for contact with the target object surface, for example, the skin of the baby for detecting health parameters of the baby. The parameters of the target object include “health parameters”, which herein comprise vital signs, for example, heart rate, body surface temperature, blood oxygen level, blood pressure, breathing rate, etc. The wearable telemetry device 100 allows remote monitoring of the health parameters. The wearable telemetry device 100 is independent of stationary power sources.

The enclosure 102 is a sealed capsule containing the telemetry device components. The enclosure 102 is, for example, about 30 mm long and 25.4 inch wide. According to one aspect of the invention, the enclosure 102 encloses a set of miniature sensors for measuring health parameters, position, motion, etc., of the target object, and a transceiver that exchanges data with external devices. The enclosure 102 is configured, for example, as a durable, waterproof, and heat resistant enclosure with complete electronic isolation for ensuring safety of the target object. The enclosure 102 is made of soft, breathable, hypoallergenic, and eco-friendly materials for further ensuring safety of the target object. The enclosure 102 is made of, for example, sanitary rubber, polyvinyl chloride, a fluoroelastomer, etc. The enclosure 102 is securely fastened within the cavity of the strap 101 of the wearable telemetry device 100 illustrated in FIG. 1A.

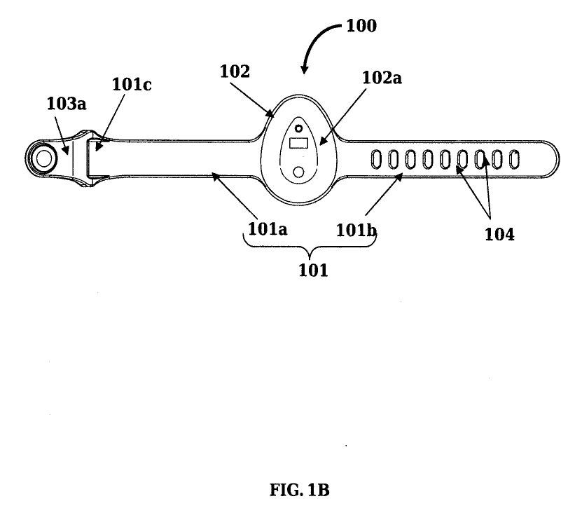

FIG. 2 exemplarily illustrates a perspective view of the wearable telemetry device 100 positioned on an arm 301 of a target object, for example, a baby. According to one modification of the invention, dimensions of the wearable telemetry device 100 are configured for positioning the wearable telemetry device 100 on a limited area of the target object's body part, for example, the baby's arm 301 for measuring the baby's vital signs.

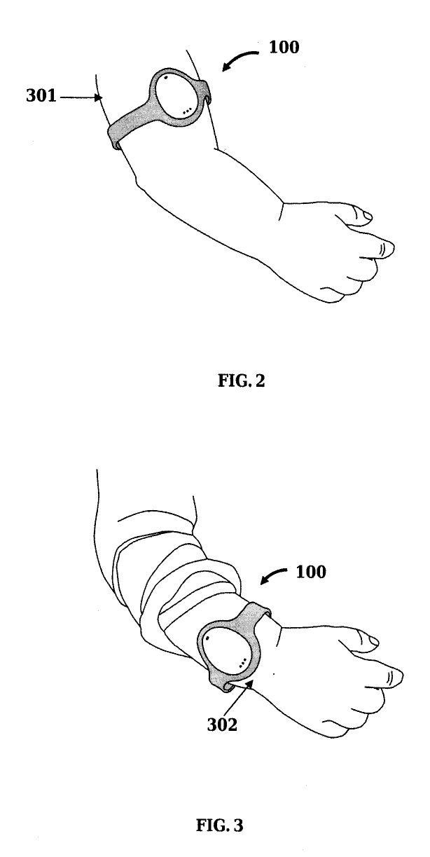

FIG. 3 exemplarily illustrates a perspective view of the wearable telemetry device 100 positioned on a wrist 302 of a target object, for example, a baby. According to one or several aspects of the invention, dimensions of the wearable telemetry device 100 are configured for positioning the wearable telemetry device 100 on a limited area of a target object's body part, for example, a baby's wrist 302, as the wrist 302 is an optimal location for positioning the wearable telemetry device 100 for measuring the baby's vital signs.

FIG. 4 exemplarily illustrates a block diagram showing the telemetry device components 400 of the wearable telemetry device 100 exemplarily illustrated in FIGS. 1A-1B. The telemetry device components 400 are electronic components positioned within the enclosure 102 of the wearable telemetry device 100 exemplarily illustrated in FIGS. FIGS. 1A-1B The telemetry device components 400 comprise at least one processor, for example, a central processing unit (CPU) 409, a wireless communication unit, e.g., a Bluetooth® transceiver module 405, multiple sensors, a codec, for example, an audio codec 410, a self-contained power source which is a rechargeable power unit, for example, a battery 401, a wireless power receiver coil 407, a wireless power charge unit 408, and a low dropout voltage regulator 402. The CPU 409 is an element on which logic connections between other telemetry device components 400 are based. The CPU 409 is, for example, the ARM® Cortex®-M4 processor of ARM Limited. The transceiver 405 is communicatively coupled to an antenna 404. The transceiver 405 performs radio wave exchange of data with external devices, for example, user devices. The transceiver 405 operates at a frequency of, for example, about 2.4 gigahertz (GHz) to about 2.4835 GHz. The transceiver 405 is configured to operate using a wireless communication protocol, for example, the Institute of Electrical and Electronics Engineers (IEEE) 802.15.1 Bluetooth® of Bluetooth Sig, Inc., or standard IEEE 802.11 Wi-Fi® of Wi-Fi Alliance Corporation. The transceiver 405 is, for example, a Bluetooth® 4.0 transceiver of Bluetooth Sig, Inc. The wearable telemetry device 100 implements software that enables connection and operation of the wearable telemetry device 100, for example, in a home network and over Public Network Internet via the transceiver 405. The transceiver 405, the sensors, the audio codec 410, the wireless power charge unit 408, and the low dropout voltage regulator 402 are operably coupled to the CPU 409 on a multilayered printed circuit board (PCB) 417. The multilayered PCB 417 serves as an assembly base for the telemetry device components 400 and conducts connections between the telemetry device components 400. Modern means of designing and manufacturing are used to manufacture the multilayered PCB 417 and integrated circuits for the wearable telemetry device 100.

The sensors are communicatively coupled to the central processing unit (CPU) 409. The sensors generate sensor data by detecting health parameters, and/or motion, and/or position of a target object. The sensors comprise, for example, a temperature sensor 403, a photoplethysmography sensor block 412a that incorporates a pulse sensor 412 and an SpO2 sensor 413, an inertial module 411a that incorporates an accelerometer such as a 3-axis accelerometer 411, a gyroscope such as a 3-axis gyroscope 414, and a magnetometer 416, a barometric pressure sensor 415, etc., or any combination thereof. The temperature sensor 403 measures body surface temperature of the target object. According to one aspect of the invention, the wearable telemetry device 100 comprises two types of temperature sensors 403, for example, a resistance thermometer and a pyrometer. The resistance thermometer operates based on a dependence of electric resistance of metals, alloys, and semiconductor materials on temperature. The pyrometer operates based on a change of power of a target object's thermal radiation in the spectra of infrared radiation and visible light. Depending on the configuration of the wearable telemetry device 100, the wearable telemetry device 100 comprises the resistance thermometer and/or the pyrometer to increase accuracy of measurements. The temperature sensor 403 allows the wearable telemetry device 100 to perform an infrared non-invasive tracking of a thermal condition of the target object based on feedback of temperature fluctuations in the upper limbs of the target object. The wearable telemetry device 100 therefore provides useful information necessary to maintain a neutral thermal environment of the target object.

The pulse sensor 412, also referred to as a “heart rate sensor”, measures a heart rate of the target object, for example, based on a phenomenon that a light signal, when passing through tissues, acquires a pulsing nature due to a change of volume of an arterial bed with each heart contraction. The pulse sensor 412 performs an optical non-invasive method of measurement of the heart rate of the target object, adapts to individual peculiarities of the target object, and filters out false readings, thereby delivering stable and accurate data. The pulse sensor 412 allows the wearable telemetry device 100 to track the heart rate of the target object in real time. Depending on the configuration of the wearable telemetry device 100, the wearable telemetry device 100 comprises one or more pulse sensors 412 to increase accuracy of measurements. The SpO2 sensor 413 measures a degree of oxygen in the blood of the target object based on the fact that absorption of light of different lengths by hemoglobin changes depending on its saturation with oxygen. The SpO2 sensor 413 performs an optical non-invasive method of measurement of oxygen levels in the blood of the target object, adapts to individual peculiarities of the target object, and filters out false readings, thereby delivering stable and accurate data. The SpO2 sensor 413 allows the wearable telemetry device 100 to expose immunodeficiency, heart disease, and respiratory problems. The wearable telemetry device 100 comprises one or more SpO2 sensors 413 to increase accuracy of measurements.

Tests were performed on the wearable telemetry device 100 to measure the accuracy of the sensors of the wearable telemetry device 100. The tests were performed on two adults of ages 26 years and 27 years. The tests composed of 15 measurements of health parameters, for example, heart rate, blood oxygen level, and temperature over a span of 3 hours. Each measurement was compared with a reference value to identify any deviations. The wearable telemetry device 100 measured the temperature of the skin surface (t° C.) of each adult as shown below:

| | Adult 1 | | | | | |
|---|---|---|---|---|---|---|
| Reference | 30.1 | 30.1 | 30 | 30.3 | 30.3 | 30.3 |
| value (t° C.) | | | | | | |
| Wearable | 30 | 30 | 30 | 30.1 | 30.1 | 30.2 |
| telemetry | | | | | | |
| device (t° C.) | | | | | | |
| Δt | 0.10 | 0.10 | 0.00 | 0.20 | 0.20 | 0.10 |

where Δt max=0.20 t° C., Δt min=−0.10 t° C., and Δt average=0.04 t° C.

| | Adult 2 | | | | | | | |
|---|---|---|---|---|---|---|---|---|
| Reference | 30.6 | 30.8 | 30.9 | 31.1 | 31.1 | 31.2 | 31.1 | 31.1 |
| value (t° C.) | | | | | | | | |
| Wearable | 30.7 | 30.8 | 30.9 | 31 | 31.1 | 31.2 | 31.1 | 31.2 |
| telemetry | | | | | | | | |
| device (t° C.) | | | | | | | | |
| Δt | 0.00 | 0.00 | 0.10 | 0.00 | 0.00 | 0.00 | −0.10 | |

where Δt max=0.20 t° C., Δt min=−0.10 t° C., and Δt average=0.04 t° C.

The wearable telemetry device 100 measured oxygen levels in the blood (%) of each adult as shown below:

| | Adult 1 | | | | | | |
|---|---|---|---|---|---|---|---|
| Reference, | 96 | 97 | 96 | 98 | 99 | 96 | 96 |
| SpO2, % | | | | | | | |
| Wearable | 96.4 | 97.2 | 97 | 97.4 | 98.2 | 96.4 | 96.4 |
| telemetry | | | | | | | |
| device, | | | | | | | |
| SpO2, % | | | | | | | |
| ΔSpO2 | −0.4 | −0.2 | −1.0 | 0.6 | 0.8 | −0.4 | −0.4 |

where ΔSpO2 max=0.8%, ΔSpO2 min=−1%, and ΔSpO2 average=0.1%.

| | Adult 2 | | | | | | |
|---|---|---|---|---|---|---|---|
| Reference, | 97 | 98 | 98 | 97 | 97 | 96 | 98 |
| SpO2, % | | | | | | | |
| Wearable | 97 | 97.4 | 97.4 | 97.3 | 97.2 | 96.2 | 97.4 |
| telemetry | | | | | | | |
| device, | | | | | | | |
| SpO2, % | | | | | | | |
| ΔSpO2 | 0 0 | 0.6 | 0.6 | −0.3 | −0.2 | −0.2 | 0.6 |

The wearable telemetry device 100 measured pulse in beats per minute (bpm) of each adult as shown below:

| | Adult 1 | | | | | | |
|---|---|---|---|---|---|---|---|
| SH-D1, P bpm | 77 | 72 | 70 | 70 | 71 | 74 | 75 |
| Wearable | 74 | 71 | 70 | 69 | 73 | 76 | 72 |
| telemetry | | | | | | | |
| device, P | | | | | | | |
| bpm | | | | | | | |
| ΔP | 3 | 1 | 0 | 1 | −2 | −2 | 3 |

where ΔP max=3 bpm, ΔP min=−2 bpm, and ΔP average=0.5 bpm.

| | Adult 2 | | | | | | |
|---|---|---|---|---|---|---|---|
| SH-D1, P | 92 | 85 | 84 | 85 | 84 | 87 | 90 |
| bpm | | | | | | | |
| Wearable | 89 | 83 | 85 | 85 | 86 | 85 | 91 |
| telemetry | | | | | | | |
| device, P | | | | | | | |
| bpm | | | | | | | |
| ΔP | 3 | 2 | −1 | 0 | −2 | 2 | −1 |

where ΔP max=3 bpm, ΔP min=−2 bpm, and ΔP average=0.5 bpm.

The 3-axis accelerometer 411 measures a projection of an apparent acceleration, that is, the difference between a true acceleration of the target object and a gravitational acceleration. The 3-axis accelerometer 411 tracks physical activity and movement of the target object. The barometric pressure sensor 415 is used to determine the absolute height required to determine the spatial orientation. The 3-axis gyroscope 414 and the magnetometer 416 determine orientation of the target object, thereby determining motion and position of the target object to monitor and analyze activities performed by the target object.

According to one aspect of the invention, the wearable telemetry device 100 further comprises an analog-to-digital converter for digitizing the sensor data collected from the sensors, for example, in the form of electrical valued. The central processing unit (CPU) 409 converts the electrical values mathematically to a form perceived by a user, creates a data packet, and sends the data packet to the user device. According to another aspect of the invention, a monitoring application is deployed on the user device for performing the mathematical reduction of the measured electrical values to save the charge of the battery 401 of the wearable telemetry device 100. According to another aspect of the invention, the telemetry device components 400 further comprise a protocol converter for converting between data transfer protocols.

The telemetry device components 400 further comprise an audio capture device, for example, a microphone 406. The microphone 406 is communicatively coupled to the central processing unit (CPU) 409. The microphone 406 receives ambient audio signals. The microphone 406 transforms sound to an analog electric signal. According to one or several aspects of the invention, the wearable telemetry device 100 transmits the analog electric signal from the microphone 406 to a direct interface user device using the transceiver 405. In another embodiment, the microphone 406 transmits the analog electric signal to the CPU 409 for performing a programmatic analysis of the analog electric signal, removing parasitic noise from the analog electric signal, and then transmitting the analog electric signal to the user device using the transceiver 405. The wearable telemetry device 100 implements a software protocol for online transmission of sound packets from the microphone 406. In one modification, the wearable telemetry device 100 comprises a highly sensitive microphone 406 coupled with a self-learning algorithm to suppress extraneous noises to transfer only sounds emitted by the target object to the user device or to the CPU 409.

The telemetry device components 400 further comprise an output device, for example, a speaker 418. The speaker 418 is communicatively coupled to the central processing unit (CPU) 409 and the transceiver 405 via the audio codec 410. The wearable telemetry device 100 implements a software protocol for online transmission of sound packets and a section in the front-end monitoring application for external devices, for example, user devices. This section of the front-end monitoring application enables control of the transmission of sound messages, playing of audio files from the memory of an external device, cloud resources, and other sources of audio Internet content. The speaker 418 transforms an electric analog signal to a sound wave. According to one aspect of the invention, the wearable telemetry device 100 comprises a miniature speaker 418 with a broad range of resonant frequencies to obtain high sound quality. The speaker 418 plays audio content, for example, from a flash memory of a remote user device or a remote interface device, for example, a smartphone, a tablet, a personal computer, a minicomputer, etc., using the transceiver 405, or from a flash memory of a local memory unit of the wearable telemetry device 100, or from a flash memory of a terminal device, for example, a base station using the transceiver 405. The speaker 418 allows a user to listen to sounds or noise made by a target object or environment in real time. The speaker 418 plays the preconfigured media data and/or the data received from the CPU 409 and/or the transceiver 405 via the audio codec 410. The audio codec 410 is used as an analog-to-digital converter of electric signals received from the microphone 406 and as a digital-to-analog converter for electric signals transmitted to the speaker 418.

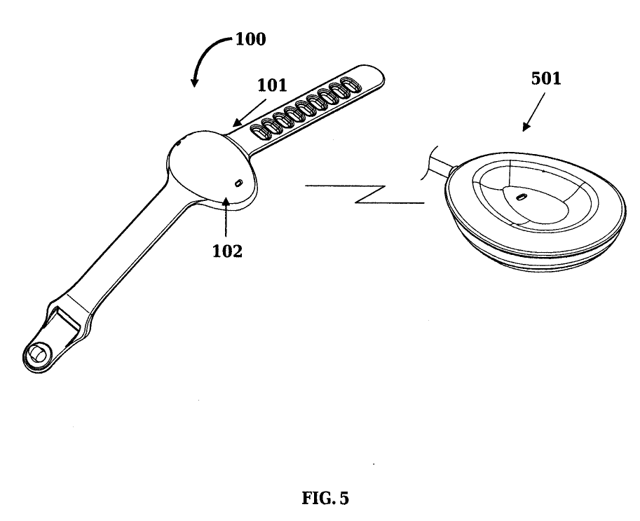

The battery 401 is an autonomous sustainable source of electric energy. The battery 401 is operably coupled to the central processing unit (CPU) 409, the transceiver 405, the sensors, for example, 403, 412, 413, 415, etc., the microphone 406, and the speaker 418 for powering the CPU 409, the transceiver 405, the sensors, the microphone 406, and the speaker 418. The wearable telemetry device 100 transmits audio tracks made by the microphone 406 and information on current charge of the battery 401 in the form of data packets to the user device. The wireless power receiver coil 407 is a receiver for wireless transmission of electric energy from the base station as disclosed in the detailed description of FIG. 5. The wireless power charge unit 408 drives the battery 401, controls the process of collection of electric energy by the wireless power receiver coil 407, and regulates a charging process of the battery 401. The wireless power charge unit 408 in electrical communication with the wireless power receiver coil 407 allows charging of the battery 401 wirelessly. The voltage regulator 402 converts voltage from the battery 401 to a level required by one or more of the telemetry device components 400. In one modification, the wearable telemetry device 100 further comprises circuit breakers (not shown) connected to each of the telemetry device components 400 for protecting the telemetry device components 400 from electrical damage, for example, by preventing overheating and/or bridging of the telemetry device components 400.

In another modification, the wearable telemetry device 100 further comprises a non-transitory computer readable storage medium, for example, a memory unit for storing the sensor data generated by the sensors, the ambient audio signals received from the microphone 406, threshold data, the preconfigured media data comprising, for example, audio data such as music, audio fairy tales, fables, poems, relaxing sounds, calming sounds, lullabies, etc., and instructions defined by modules of the wearable telemetry device 100. In s, the wearable telemetry device 100 updates the audio data automatically based on the age of the target object. The central processing unit (CPU) 409 is communicatively coupled to the non-transitory computer readable storage medium and operably coupled to the transceiver 405. The transceiver 405 receives data packets with settings and commands from the user device and transmits the data packets to the CPU 409 of the wearable telemetry device 100. The CPU 409 executes the commands and one or more computer program instructions defined by the modules of the wearable telemetry device 100, collects information from the sensors in the set mode, and controls operation of the speaker 418 and the microphone 406.

The modules of the wearable telemetry device 100 comprise a data communication module, a control module, a data processing module, and an alerting module 404a. The data communication module receives user input data from the monitoring application deployed on one or more user devices via the transceiver 405 and facilitates communication between the target object and the user devices via the transceiver 405. The user input data comprises, for example, modes for activating and controlling operability and sensitivity of the sensors, the microphone 406, and the speaker 418, safe areas, spots dangerous for the target object, tasks to be accomplished, customizations for alert notifications, etc. The monitoring application is configured, for example, as a mobile application or a desktop application. The monitoring application can be deployed on user devices that execute operating systems of different types, for example, the Android® operating system of Google Inc., the iOS operating system of Apple Inc., the Windows Phone® operating system of Microsoft Corporation, etc. The control module activates and controls operability and sensitivity of the sensors, and/or the microphone 406, and/or the speaker 418 based on the received user input data. The control module activates and deactivates the microphone 406 and the speaker 418 based on instructions received from the user device via a graphical user experience presentation screen (GUI) provided by the monitoring application deployed on the user device. The control module receives a selection of modes of operation of the wearable telemetry device 100, for example, the modes of operation of the sensors, and activates the sensors to measure one or more health parameters of the target object. The modes of operation comprise, for example, activation of the sensors for measurement and updating of the health parameters of the target object with a certain regularity, activation of the sensors for measurement and updating of the health parameters of the target object based on a measurement request received from the monitoring application deployed on the user device, and activation of the sensors for measurement and transmitting the health parameters of the target object to the user device in real time.

It can be seen from FIG. 4 that the CPU 409 incorporates a number of various program functional modules such as a sound control module 1001, a spatial orientation module 1003, a heart rate and SPO2 measurement control module 1005, a data transmitter/receiver module 1007, a temperature measurement control module 1009, an absolute height determination module 1011, a comparison with signal pattern module 1013, a wavelength generation module 1015, a vibration determination module 1017, as well as a DC filter 1019, an ambient lighting filter 1021, an a data validation module 1018. The sound control module 1001 is linked to the data/transmitter/receiver module 1007, and the latter is connected to the antenna 404 via the Bluetooth® transceiver module 405. The spatial orientation module 1003 is linked to the 3-axis accelerometer 411, the 3-axis gyroscope 414, and the 3-axis magnetometer 416 of the inertial module 411a. The PGG sensor block photoplethysmography 412a sensor unit is a combination of two sensors: a heart sensor 412 and a SPO2 sensor 413. It also incorporates a photodiode 1023, an infrared LED 1025, a red LED 1027, and a green LED 1029. The photodiode 1023 is linked to the ambient light filter 1021 via an A/D converter 1031. Other links and relationships between various sensors, modules, and units of the telemetry device components 400 are shown in FIG. 4.

An example of a photoplethysmography sensor block that incorporates a red LED, a green LED, and photodiode is a sensor SFH 7050 of the Health Monitoring Series produced by OSRAM Opto Semiconductors GmbH (Germany).

When a light wave, emitted by the green LED, is reflected from the blood tissue (capillaries, blood vessels, etc.) at different stages of their contraction, this changes intensity of the transmitted light, and when the reflected light falls on the photodiode, the latter generates a current which is directly proportional to the light intensity and, hence, to variations/contractions of the blood vessels. The frequency of such variations/contractions corresponds to the heart rate of the target object. The effect of the extraneous ambient and/or artificial light is reduced or eliminated by means of appropriate filters. Pulse oximetry is based on the principle that O₂Hb and HHb differentially absorb red and near-infrared (IR) light. In case of the SPO2 sensor, red and infrared LEDs that emit light shining through a reasonably translucent skin of a baby are used. Oxygenated hemoglobin absorbs more infrared light and allows more red light to pass through. Deoxygenated (or reduced) hemoglobin absorbs more red light and allows more infrared light to pass through. Red light is in the range of 600-750 nm wavelength light band. Infrared light is in the range of 850-1000 nm wavelength light band. The photodetector receives the light that passes through the measuring site. The data processing module filters and processes the sensor data generated by the activated sensors and computes monitoring indices by processing the detected health parameters, the motion, and/or the position of the target object from the filtered and processed data in accordance with the threshold data, and/or the received ambient audio signals, and/or the received user input data. As used herein, “monitoring indexes” refers to health indexes, for example, an average heart rate, oxygen level in the blood, temperature, etc. The alerting module generates alert notifications in multiple formats, for example, audio alerts, video alerts, text messages, etc., based on the computed monitoring indexes and transmits the generated alert notifications to the target object via the wearable telemetry device 100 and/or the user devices via the Bluetooth® transceiver module 405. For example, if temperature readings of a target object such as a baby deviate from a normal temperature, the alerting module alerts a user by generating and transmitting alert notifications, for example, in the form of a sound or a visual signal. The wearable telemetry device 100 implements remote firmware update technology that allows updating of the firmware of the wearable telemetry device 100 remotely.

FIG. 5 exemplarily illustrates wireless communication between the wearable telemetry device 100 and a base station 501. According to one or several aspects of the invention, the rechargeable power unit, for example, the battery 401 exemplarily illustrated in FIG. 4, of the wearable telemetry device 100 is operably coupled to the base station 501 to allow the base station 501 to wirelessly transmit power from an external power source to the battery 401 of the wearable telemetry device 100. The base station 501 is implemented, for example, as an inductive charger station and operates with wireless communication technology, for example, Bluetooth 4.0 technology and a Wi-Fi® module. The base station 501 is a source of wireless transmission of electric energy for wirelessly charging the wearable telemetry device 100. The wearable telemetry device 100 has to be charged regularly. The life of the battery 401 of the wearable telemetry device 100 is, for example, from about 48 hours to up to about 30 days without heavy use of the speaker 418 exemplarily illustrated in FIG. 4. According to another aspect of the invention, the battery 401 of the wearable telemetry device 100 is charged wirelessly from the base station 501 by an inductive transmission of electric energy between two coils. The base station 501 is powered from the mains power supply. In another embodiment, the battery 401 of the wearable telemetry device 100 is charged by wired charging, for example, using a mains voltage converter.

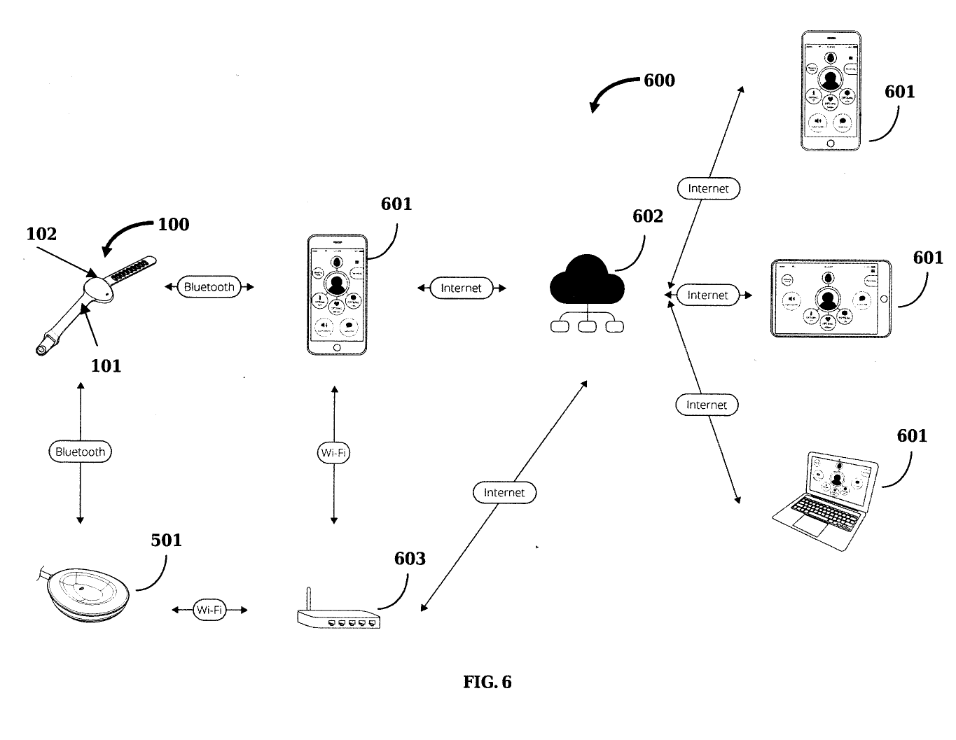

FIG. 6 exemplarily illustrates a system 600 for monitoring a target object, showing wireless communication between the wearable telemetry device 100, the base station 501, and multiple user devices 601, for example, a laptop, a smartphone, a tablet computer, etc. The wireless communication is implemented using a combination of wireless communication protocols, for example, a Bluetooth® communication protocol, a Wi-Fi® communication protocol, etc. In one modification, the wearable telemetry device 100 is combined with a converter of wireless communication protocols for converting between, for example, a Bluetooth® data transfer protocol and a Wi-Fi® data transfer protocol. As exemplarily illustrated in FIG. 6, the wearable telemetry device 100 communicates with the base station 501 and one or more user devices 601, for example, via the Bluetooth® data transfer protocol. The wearable telemetry device 100 positioned on a forearm 301 or a wrist 302 exemplarily illustrated in FIGS. 3A-3B, of a target object, for example, a baby is configured to communicate with telemetry and multimedia systems. The wearable telemetry device 100 operates directly with a user device 601, for example, a smartphone using a Bluetooth® channel or a Wi-Fi® channel, provided there is a wireless network in the operation area. One or more user devices 601 can connect to the wearable telemetry device 100 using point-to-point and/or point-to-multipoint connections via one or more data transfer or wireless communication protocols. The base station 501 communicates with a router, for example, a Wi-Fi® router 603 via the Wi-Fi® data transfer protocol. The Wi-Fi® router 603 communicates with the user device 601 via the Wi-Fi® data transfer protocol, and with cloud resources 602 via a network, for example, the Internet. Multiple user devices 601 access the cloud resources 602 via the Internet. ‘ The wearable’ telemetry device 100 enables remote communication between a target object and users in close proximity to the wearable telemetry device 100 through the monitoring application deployed on each of the user devices 601. Interchangeable connection protocols allow the wearable telemetry device 100 to interact with multiple user devices 601 at any distance while at least one device with an Internet connection, including the base station 501, is in a range of, for example, 100 meters.

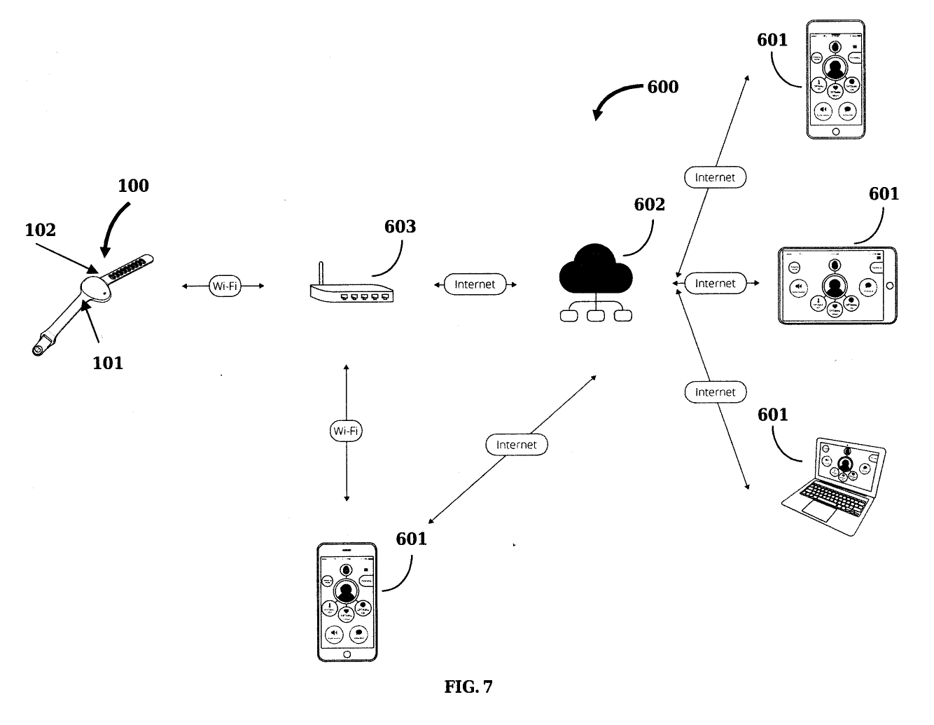

FIG. 7 exemplarily illustrates an embodiment of the system 600 for monitoring a target object, showing wireless communication between the wearable telemetry device 100 and multiple user devices 601. As exemplarily illustrated in FIG. 7, the wearable telemetry device 100 communicates with the user device 601, for example, via a router, for example, a Wi-Fi® router 603 using the Wi-Fi® data transfer protocol. The Wi-Fi® router 603 and the user device 601 then access cloud resources 602 via a network, for example, the Internet. Multiple user devices 601 access the cloud resources 602 via the Internet.

Consider an example where a user, for example, a parent attaches the wearable telemetry device 100 to a baby's arm 301 or wrist 302 exemplarily illustrated in FIGS. 3A-3B, using the strap 101 of the wearable telemetry device 100. Using a setup panel on a graphical user interface provided by the monitoring application of the user device 601, the user selects one of the modes of operation of the wearable telemetry device 100 comprising, for example, the pulse or heart rate sensors 412, the SpO2 sensors 413, and the temperature sensors 403 exemplarily illustrated in FIG. 4. The modes of operation of the wearable telemetry device 100 comprise: Mode 1: Automatic reading of the sensors with a certain regularity; Mode 2: reading of the sensors on request of the user; and Mode 3: measuring and transmission of readings to the user device 601 in real time. Using the same user device 601, the user can enable or disable the microphone 406 of the wearable telemetry device 100 exemplarily illustrated in FIG. 4, to listen to or mute external sounds from the microphone 406, enable or disable audio playback by the speaker 418 of the wearable telemetry device 100 exemplarily illustrated in FIG. 4, and adjust the sensitivity of the microphone 406 and the speaker 418. The wearable telemetry device 100 enables playback of different types of media content comprising, for example, music, audio fairy tales, fables, poems, relaxing sounds, calming sounds, lullabies, etc., via the speaker 418. On performance of the above actions, the monitoring application on the user device 601 creates data packets instantly and transmits the created data packets to the wearable telemetry device 100. The monitoring application on the user device 601 transmits the data packets to the wearable telemetry device 100 by different methods. In one modification, data is transmitted directly between the wearable telemetry device 100 and the user device 601, for example, by the Bluetooth® data transfer protocol as exemplarily illustrated in FIG. 6. In another embodiment, the data is transmitted indirectly between the wearable telemetry device 100 and the base station 501, for example, by the Bluetooth® data transfer protocol and then by means of a home network segment, for example, the Wi-Fi® router 603 to the user device 601 as exemplarily illustrated in FIG. 6. In both embodiments above, in case of access of the user device 601 or the Wi-Fi® router 603 to the Internet, data is stored in the cloud resources 602 and is available to other user devices 601.

In another embodiment as exemplarily illustrated in FIG. 7, data is transmitted between the wearable telemetry device 100 and the user device 601 using an access point created in a Wi-Fi® operation area, for example, the Wi-Fi® router 603. In case of access of the user device 601 or the Wi-Fi® router 603 to the Internet, data can be stored in the cloud resources 602 and is made available to other user devices 601. The system 600 implements a packet exchange protocol that enables the system 600 to select a path to a user device 601 and the cloud resources 602 if there are no network elements. If there are several paths, the packet exchange protocol selects the shortest path.

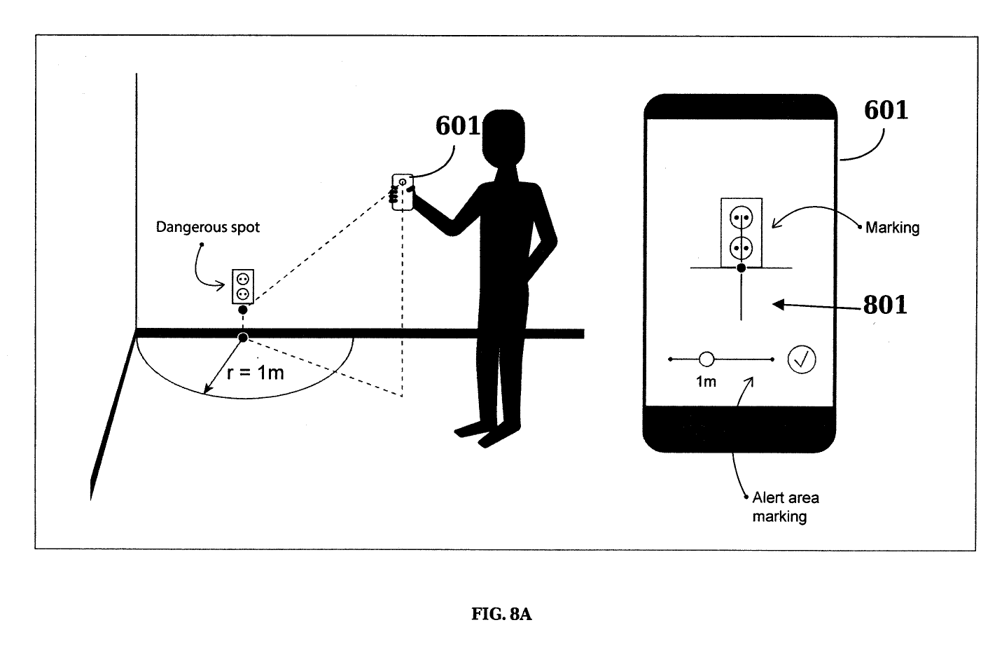

FIGS. 8A-8B exemplarily illustrate configuration of dangerous spots 804 in a region using the monitoring application deployed On a user device 601 for monitoring a target object. A user marks dangerous spots 804 via a graphical user experience presentation screen (GUI) 801 provided by the monitoring application deployed on a user device 601 as exemplarily illustrated in FIGS. 8A-8B. The GUI 801 displays information and controls for use by the user.

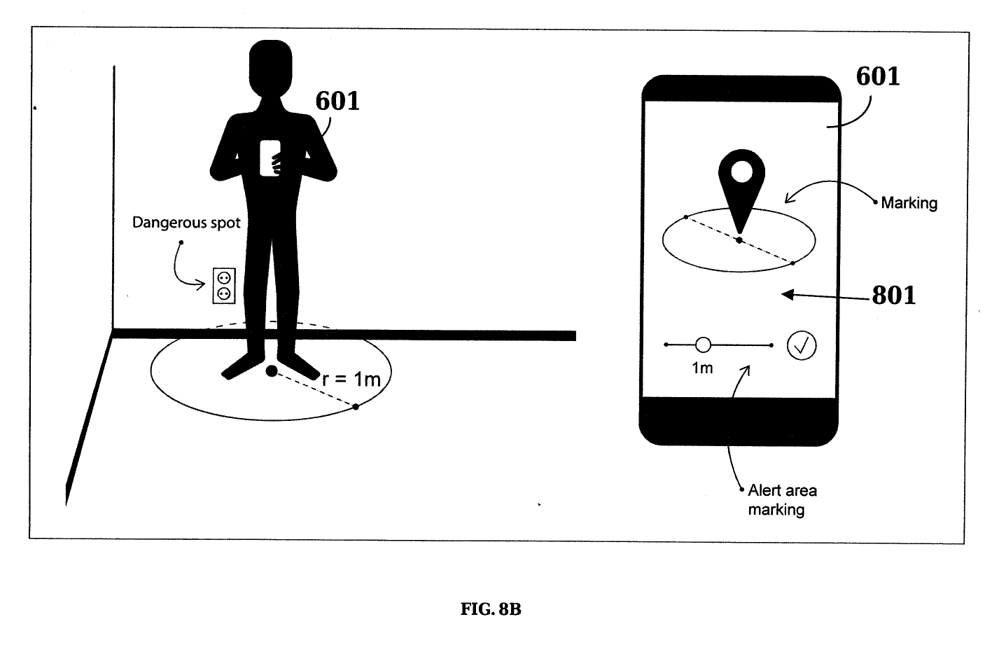

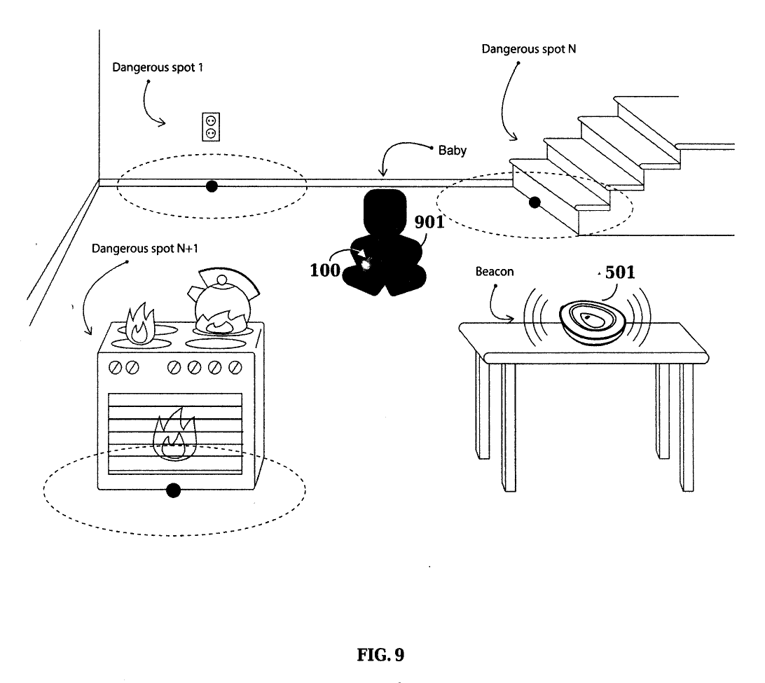

FIGS. 9-10 exemplarily illustrate monitoring of a target object, for example, a baby 901 in an environment with dangerous spots 902a, 902b, and 902c. identified using the monitoring application deployed on a user device 601 as exemplarily illustrated in FIGS. 8A-8B. FIG. 9 exemplarily illustrates one more dangerous spot surrounding the baby 901. The wearable telemetry device 100 and, in modification of the device, one or more beacons track the baby's 901 movement and produce visual and sound notifications on the user device 601 if the baby 901 approaches any of these dangerous spots. The wearable telemetry device 100, in communication with the monitoring application deployed on the user device 601, performs the tracking using an inertial navigation system (INS). The base station 501 is used as a coordinate origin to mark the dangerous spots. The wearable telemetry device 100 monitors the position of the baby 901 to detect dangerous or potentially dangerous situations. The wearable telemetry device 100 tracks the baby 901 in a three-axis coordinate system relative to the coordinate origin based on metrics of the 3-axis accelerometer 411 and the 3-axis gyroscope 414 exemplarily illustrated in FIG. 4, as a part of the INS, and a compass such as the magnetometer 416 exemplarily illustrated in FIG. 4, and a barometer such as a barometric pressure sensor 415 exemplarily illustrated in FIG. 4. According to one aspect of the invention, the wearable telemetry device 100 and the monitoring application of the user device 601, as exemplarily illustrated in FIG. 10, are used to mark the area around the baby's 901 current location and send visual and sound alert notifications to the user device 601 if the baby 901 leaves the marked area. Using the monitoring application on the user device 601, the user resets coordinates of the wearable telemetry device 100 at a desired location and marks a “safe” area around the desired location.

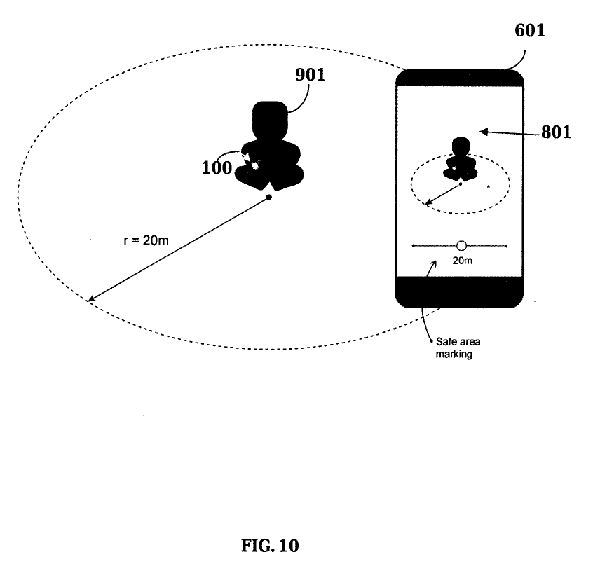

FIG. 11 exemplarily illustrates a flowchart comprising the steps for determining a heart rate of a target object, for example, a baby 901 exemplarily illustrated in FIGS. 9-10, using the heart-rate output data determination and correlation block 1033 of the wearable telemetry device 100 exemplarily illustrated in FIGS. 1A-1C, worn by the baby 901. The wearable telemetry device 100 is based on an optical non-invasive measurement technology. The method of measurement of heart rate is based on a phenomenon that light signal, when passing through tissues, acquires a pulsing nature due to a change of volume of an arterial bed with each heart contraction. A user, for example, a parent, places 1101 the wearable telemetry device 100 comprising the pulse sensor 412 exemplarily illustrated in FIG. 4, or a heart rate sensor on the baby's 901 wrist 302 exemplarily illustrated in FIG. 3B. The wearable telemetry device 100 enables 1102 a green light emitting diode (LED) based on instructions received from the user via the GUI provided by the monitoring application deployed on the user device 601 exemplarily illustrated in FIGS. 6-7, for example, a smartphone. The wearable telemetry device 100 receives 1103 data comprising, for example, reflected light component, one or more ambient light components, direct current (DC) components, etc., from a photodiode of the wearable telemetry device 100. The wearable telemetry device 100 filters the received data by removing 1104 ambient light components from the received data. The wearable telemetry device 100 also filters the received data by removing 1105 the DC components from the received data. The result data contains the change of light, absorbed by blood, and gives the information about blood pulsations. The wearable telemetry device 100 then determines 1106 external vibrations based on data analysis received from the 3-axis gyroscope 414 exemplarily illustrated in FIG. 4 and the 3-axis accelerometer 411 exemplarily illustrated in FIG. 4 from the baby's 901 wrist 302 exemplarily illustrated in FIG. 3B. The wearable telemetry device 100 then determines 1107 the filtered HR data reliability based on the determined external vibrations and if the filtered HR data is not fully reliable 1108, the wearable telemetry device 100 then excludes unreliable data and estimates 1109 actual data using wavelet transformation. The wearable telemetry device 100 applies 1110 the special signal pattern to the reliable data and estimated data and calculates the correlation 1111 of the pattern signal and data to do it more suitable for further calculations. The resultant correlation data is used by the wearable telemetry device 100 to calculate 1112 a monitoring index, for example, an average heart rate value. The wearable telemetry device 100 measures and monitors the baby's 901 heart rate with medical quality accuracy.

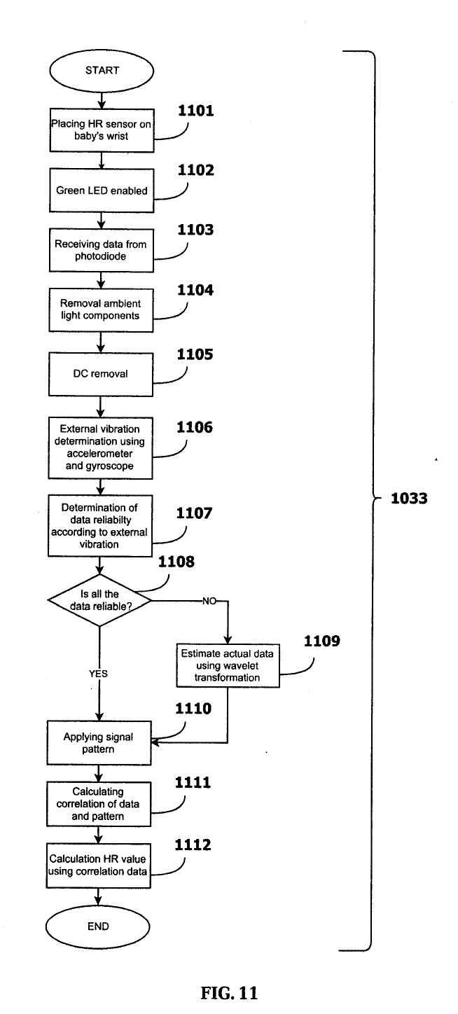

FIG. 12 exemplarily illustrates a flowchart comprising the steps for determining an oxygen saturation level in the blood of a target object, for example, a baby 901 exemplarily illustrated in FIGS. 9-10, using the output SPO2 value, determination and correlation block 1035 of the wearable telemetry device 100 exemplarily illustrated in FIGS. 1A-1C, worn by the baby 901. The method of measurement of the oxygen level in the blood is based on the fact that the absorption of light of two different lengths by hemoglobin changes depending on the saturation level of oxygen in the blood. A user, for example, a parent, places 1201 the wearable telemetry device 100 comprising the oxygen saturation level (SpO2) sensor 413 exemplarily illustrated in FIG. 4, on the baby's 901 wrist 302 exemplarily illustrated in FIG. 3B. The wearable telemetry device 100 enables 1202 a red light emitting diode (LED) and an infrared (IR) LED based on instructions received from the user via a graphical user experience presentation screen (GUI) provided by the monitoring application deployed on a user device 601 exemplarily illustrated in FIGS. 6-7, for example, a smartphone. The wearable telemetry device 100 receives 1203 a red signal and an infrared signal comprising alternating current (AC) components, direct current (DC) components, etc., from a photodiode. The wearable telemetry device 100 filters the received data by removing 1204 ambient light components from the received data. The wearable telemetry device 100 then determines 1205 external vibrations based on data analysis received from the 3-axis gyroscope 414 exemplarily illustrated in FIG. 4 and the 3-axis accelerometer 411 exemplarily illustrated in FIG. 4 from the baby's 901 wrist 302 exemplarily illustrated in FIG. 3B. The wearable telemetry device 100 then determines 1206 the SpO2 data reliability based on the determined external vibrations and if the SpO2 data is not fully reliable 1207, the wearable telemetry device 100 then excludes unreliable data and estimates 1208 actual data using wavelet transformation. The wearable telemetry device 100 determines 1209 an AC component of the received red signal and IR signal. The wearable telemetry device 100 determines 1210 a DC component of the received red signal and IR signal. The wearable telemetry device 100 calculates 1211 a monitoring index, for example, the value of SpO2 in the blood circulation. The calculations are based on determination of AC and DC ratio of red signal and IR signal. The wearable telemetry device 100 measures and monitors SpO2 indexes with medical quality accuracy. The wearable telemetry device 100 implements an optical non-invasive measurement technology.

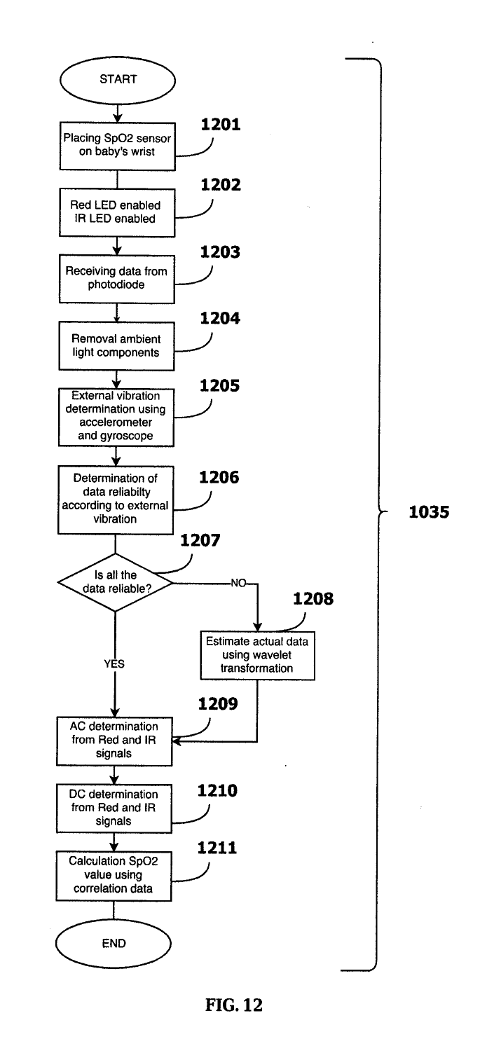

FIG. 13 exemplarily illustrates a flowchart comprising the steps for determining a reference point to set a temperature extreme of thermal comfort of a target object, for example, a baby 901 exemplarily illustrated in FIGS. 9-10, using the wearable telemetry device 100 exemplarily illustrated in FIGS. 1A-1C, worn by the baby 901. A user, for example, a parent places the wearable telemetry device 100 comprising the temperature sensor 403 exemplarily illustrated in FIG. 4, on the baby's 901 wrist 302 exemplarily illustrated in FIG. 3B. The temperature sensor 403 measures 1301 the temperature of the baby 901 on the skin of the baby's 901 upper extremity based on instructions received from the user via a graphical user experience presentation screen (GUI) provided by the monitoring application deployed on a user device 601 exemplarily illustrated in FIGS. 6-7, for example, a smartphone.

The wearable telemetry device 100 determines 1302 whether the measured result is in the range of optimal temperatures. If the measured result is in the range of optimal temperatures, then the wearable telemetry device 100 renders 1303 the optimal temperature information to the user device 601 for confirmation. The wearable telemetry device 100 checks 1305 for user confirmation. If the wearable telemetry device 100 receives a user confirmation from the user device 601, then the wearable telemetry device 100 uses 1309 the measured temperature result as a reference point to set a temperature extreme of thermal comfort. If the wearable telemetry device 100 does not receive a user confirmation from the user device 601, then the wearable telemetry device 100 allows a user to perform the measurement 1307 in more favorable conditions and continues to measure 1301 the temperature. If the temperature measured is not in the range of the optimal temperatures, then the wearable telemetry device 100 renders 1304 the non-optimal temperature information to the user device 601 for confirmation and checks 1306 for user confirmation. If the wearable telemetry device 100 receives a user confirmation from the user device 601, then the wearable telemetry device 100 allows the user to perform the measurement 1308 in more favorable conditions and continues to measure 1301 the temperature. If the wearable telemetry device 100 does not receive a user confirmation from the user device 601, then the wearable telemetry device 100 uses 1309 the measured temperature result as a reference point to set a temperature extreme of thermal comfort. The wearable telemetry device 100 measures and monitors the baby's 901 thermal comfort to provide the user with data needed to maintain the baby's 901 neutral thermal environment. The measurement technology is based on a human physiological function, when vasoconstriction and vasodilatation vary blood flow to the baby's 901 hands and other extremities to control heat loss from the baby's 901 skin to the environment. As a result, cold hands indicate that the baby's 901 body is acting to retain heat, while warm hands indicate the baby's 901 body is acting to lose heat. The wearable telemetry device 100 implements an infrared temperature measurement technology and an algorithm comprising, for example, two main stages. For example, the first stage is initial measurement, which is made to determine the wrist 302 or forearm's 301 temperature extreme of thermal comfort state of the baby 901. The second stage relates to consequent measurements, which are made to monitor temperature deviations affecting the baby's 901 thermal comfort.

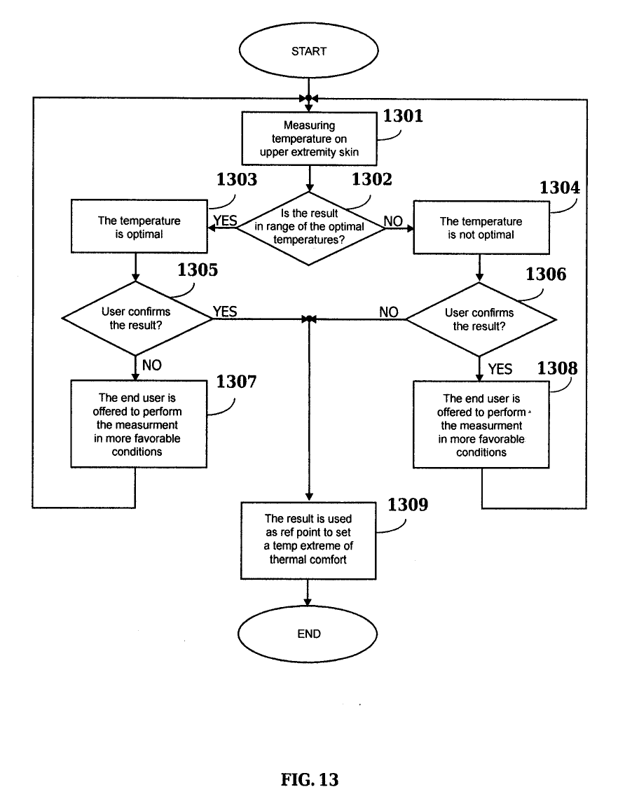

FIG. 14 exemplarily illustrates a flowchart comprising the steps for reproducing sound of a target object, for example, a baby 901 exemplarily illustrated in FIGS. 9-10, on a user device 601 exemplarily illustrated in FIGS. 6-7, using the wearable telemetry device 100 exemplarily illustrated in FIGS. 1A-1C, worn by the baby 901. The wearable telemetry device 100 receives 1401 an ambient sound frame from the microphone 406 exemplarily illustrated in FIG. 4, based on instructions received from the user via a graphical user experience presentation screen (GUI) provided by the monitoring application deployed on the user device 601. The wearable telemetry device 100 processes 1402 the received sound frame using a high pass filter in the wearable telemetry device 100 to remove high frequency extraneous noise from the received sound frame. The wearable telemetry device 100 processes 1403 the high pass filtered sound frame using a low pass filter to remove low frequency extraneous noise from the sound frame. The wearable telemetry device 100 suppresses 1404 background noise from the low pass filtered sound frame.

The wearable telemetry device 100 performs echo cancellation 1405 on the background noise suppressed sound frame. The wearable telemetry device 100 performs an audio analytics comparison 1406 of frequencies of the echo cancelled sound frame to detect 1407 the sound emitted by the baby 901. If the wearable telemetry device 100 detects the sound emitted by the baby 901, then the wearable telemetry device 100 wirelessly transmits 1408 the detected data of the sound frame to the user device 601 to reproduce 1409 the sound of the baby 901 on the user device 601, for example, via a Wi-Fi® communication protocol or a Bluetooth® communication protocol. If the wearable telemetry device 100 is unable to detect the sound emitted by the baby 901, the wearable telemetry device 100 receives 1401 the sound frame again from the microphone 406 for further processing and detection. The wearable telemetry device 100 also performs programmatic analysis of the received sound frame from the microphone 406. The wearable telemetry device 100 removes extraneous noise from the received sound frame to monitor the sound produced only by the baby 901.

According to one aspect of the invention, the wearable telemetry device 100 tracks and analyzes a sleep pattern of the baby 901. In this embodiment, the wearable telemetry device 100 performs an analysis on quality of sleep and stages of sleep. The wearable telemetry device 100 processes and analyses the data received from the microphone 406 and the 3-axis accelerometer 411 exemplarily illustrated in FIG. 4. The wearable telemetry device 100 detects whether the baby 901 is awake and sends visual and sound alert notifications to the user device 601 to alert the user. Limiting the audio signal perception area of the microphone 406 by implementing a noise cancellation algorithm provides improved results. The microphone 406 is highly sensitive and implements an extraneous noise suppression technology. The microphone 406 is coupled with a self-learning algorithm to suppress extraneous noise to transfer sounds emitted by the baby 901 to the user device 601. The wearable telemetry device 100 enables remote voice communication between the baby 901 and the user device 601. The wearable telemetry device 100 implements interchangeable connection protocols for interaction with user devices 601 at any distance from the wearable telemetry device 100, while at least one device, for example, the base station 501 is in a range of, for example, a 100 meters, thereby allowing users to communicate with the target object from any location at any time. The wearable telemetry device 100 reproduces a variety of audio content accessed through the user device 601.

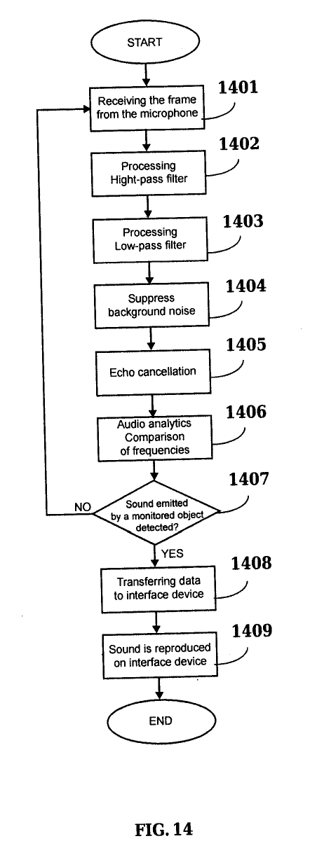

FIG. 15 exemplarily illustrates a flowchart comprising the steps for initiating wireless transmission of sensor data to a user device 601 exemplarily illustrated in FIGS. 6-7, or the base station 501 exemplarily illustrated in FIG. 5, proximal to the wearable telemetry device 100 exemplarily illustrated in FIGS. 1A-1C, via a wireless communication protocol based on a deviation found in the sensor data. The delivery of data packets by the wearable telemetry device 100 depends upon the proximity of the base station 501 and the user device 601. The wearable telemetry device 100 receives one or more sensor readings based on instructions received from the user via a graphical user experience presentation screen (GUI) provided by the monitoring application deployed on the user device 601. The wearable telemetry device 100 checks 1501 whether a first measurement of the sensor data was made. If the first measurement was not made, the wearable telemetry device 100 measures 1502 the sensor data, for example, health parameters of the target object, and computes monitoring indexes from the measured sensor data. The wearable telemetry device 100 determines 1503 whether there are deviations of the computed monitoring indexes from the data measured previously. If a deviation is not found, the wearable telemetry device 100 determines 1501 whether the current measurement is the first measurement. If the current measurement is not the first measurement, then the wearable telemetry device 100 continues to measure 1502 the sensor data and check 1503 for a deviation. If the wearable telemetry device 100 finds a deviation or if the current measurement is the first measurement made by the wearable telemetry device 100, the wearable telemetry device 100 is programmed to transmit the sensor data.

The wearable telemetry device 100 enables 1504 a Bluetooth® communication protocol therewithin to transmit the measured sensor data. The wearable telemetry device 100 enables the Bluetooth® communication protocol to initiate transfer of the measured sensor data only when a deviation is detected. The wearable telemetry device 100 searches 1505 for devices to pair with for transmitting the measured sensor data. The wearable telemetry device 100 checks 1506 whether an interface device, that is, a user device 601 is found. If a user device 601 is found, then the wearable telemetry device 100 determines 1507 the proximity of the user device 601 from the wearable telemetry device 100 and checks 1509 for the presence of a terminal, that is, the base station 501. If the base station 501 is found, then the wearable telemetry device 100 determines 1510 the proximity of the base station 501 from the wearable telemetry device 100. The wearable telemetry device 100 checks 1511 whether at least one device is found. If the user device 601 and the base station 501 are not found, the wearable telemetry device 100 waits for a predetermined time delay 1508, enables 1504 the Bluetooth® communication protocol again, and continues to search 1505 for the user device 601 and the base station 501.

If the wearable telemetry device 100 finds at least one of the user device 601 and the base station 501, the wearable telemetry device 100 compares 1513 the proximity of the user device 601 from the wearable telemetry device 100 with the proximity of the base station 501 from the wearable telemetry device 100. If the proximity of the user device 601 with respect to the wearable telemetry device 100 is greater than the proximity of the base station 501 with respect to the wearable telemetry device 100, then the wearable telemetry device 100 connects 1514 to the base station 501 and transmits 1516 the measured sensor data to the base station 501. If the proximity of the user device 601 with respect to the wearable telemetry device 100 is lesser than the proximity of the base station 501 with respect to the wearable telemetry device 100, then the wearable telemetry device 100 connects 1512 to the user device 601 and transmits 1515 the measured sensor data to the user device 601. The wearable telemetry device 100 determines the device closest to the wearable telemetry device 100 to decrease electromagnetic radiation (EMR) during transmission of the measured sensor data. On transmitting the measured sensor data, the wearable telemetry device 100 determines 1517 whether the measured sensor data has been sent successfully. If the wearable telemetry device 100 sends the measured sensor data successfully, the wearable telemetry device 100 continues with the measurement of the sensor data. If the wearable telemetry device 100 did not send the measured sensor data successfully, the wearable telemetry device 100 enables 1504 the Bluetooth® communication protocol again to search 1505 for devices for transmitting the measured sensor data. The wearable telemetry device 100 substantially or completely removes potentially harmful EMR and electromagnetic frequencies (EMF) during the use of the wearable telemetry device 100. As a result of a combination of autonomous operation and a data exchange algorithm, the wearable telemetry device 100 does not emit high or chronic electromagnetic radiation. The wearable telemetry device 100 combines the autonomous operation, the data exchange algorithm, and a Bluetooth® low energy (BLE) technology. The BLE technology is enabled only if there is sensitive data that requires the user's attention. The monitoring application deployed on the user device 601 displays the measured sensor data sent by the wearable telemetry device 100, for example, as a visual representation on the graphical user interface on the user device 601.

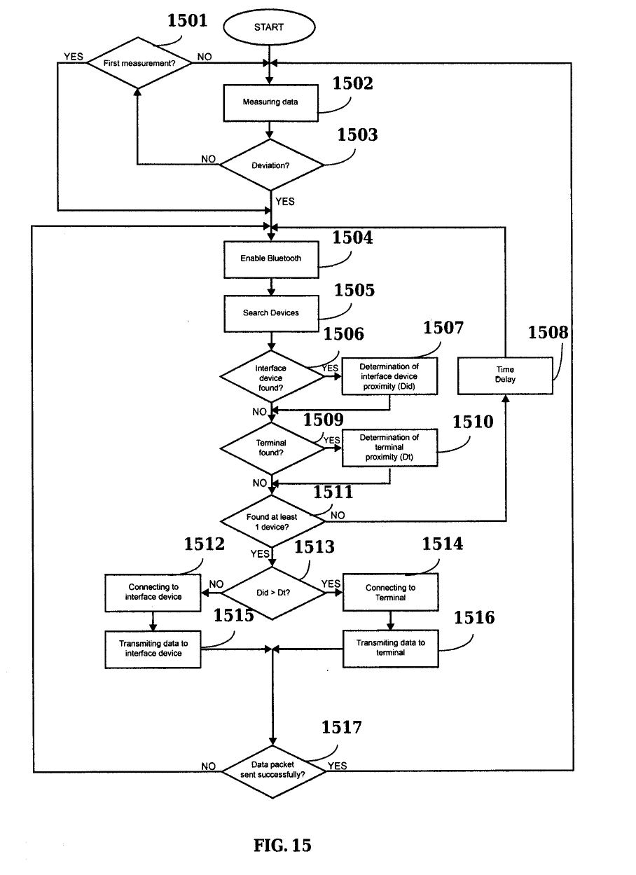

FIG. 16 exemplarily illustrates a flowchart comprising the steps for transmitting alert notifications to a user device 601 exemplarily illustrated in FIGS. 6-7, or the base station 501 exemplarily illustrated in FIG. 5, on determining a deviation of measured data from threshold data. The wearable telemetry device 100 exemplarily illustrated in the FIGS. 1A-1C, measures 1601 health parameters of a target object, for example, a baby 901 exemplarily illustrated in FIGS. 9-10. The wearable telemetry device 100 checks 1602 for deviations in the measured data with respect to the threshold data. If deviations are not found, the wearable telemetry device 100 continues to measure 1601 the health parameters of the baby 901. If deviations are found, the wearable telemetry device 100 generates and transmits 1603 visual and sound alerts to an interface device, that is, the user device 601. The wearable telemetry device 100 waits for a predetermined time delay 1604 after transmitting the visual and sound alerts to the user device 601. The wearable telemetry device 100 checks 1605 whether the visual and sound alerts have been delivered to the user device 601 successfully. If the visual and sound alerts have been delivered successfully, the wearable telemetry device 100 continues to measure 1601 the health parameters of the baby 901. If the visual and sound alerts have not been delivered successfully, the wearable telemetry device 100 generates and transmits 1606 a sound alert to a terminal, that is, to the base station 501. The wearable telemetry device 100 waits for a predetermined time delay 1607 after transmitting the sound alert to the base station 501. The wearable telemetry device 100 checks 1608 whether the sound alert has been delivered successfully. If the sound alert has been delivered successfully, the wearable telemetry device 100 continues to measure 1601 the health parameters of the baby 901.

If the sound alert has not been delivered to the base station 501 successfully, the wearable telemetry device 100 generates and transmits 1609 a sound alert to the wearable telemetry device 100. After a predetermined time delay 1610 after generating the sound alert, the wearable telemetry device 100 checks 1611 whether the sound alert has been delivered successfully. If the sound alert has been delivered successfully, the wearable telemetry device 100 continues to measure 1601 the health parameters of the baby 901. If the sound alert has not been delivered successfully, the wearable telemetry device 100 generates 1603 visual and sound alerts in the user device 601. The method disclosed herein implements a 3-stage notification delivery system to guarantee emergency alert delivery to the user. If an emergency alert cannot be delivered to the monitoring application deployed on the user device 601 under certain circumstances or if the emergency alert was missed by the user, an alert sound is played on the base station 501. If the base station 501 cannot play the alert sound, the sound alert is produced on the wearable telemetry device 100. The monitoring application on the user device 601 allows a user to customize the alert notifications, assign an emergency alert status, and enable or disable the alert notifications for certain events via the graphical user interface.

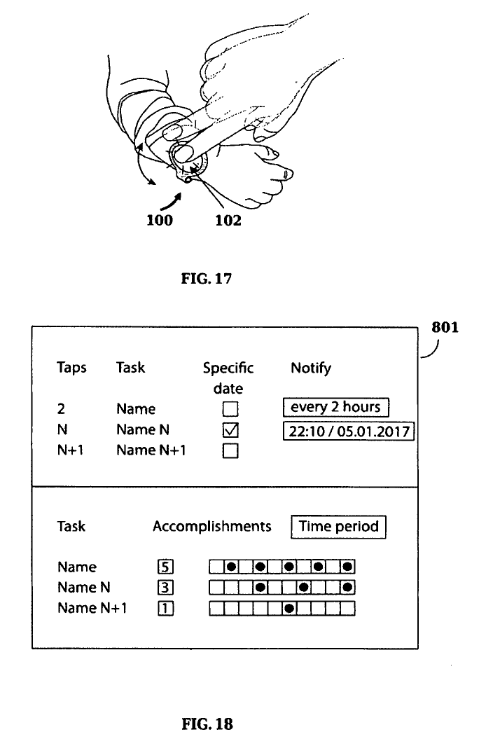

FIG. 17 exemplarily illustrates confirmation of task accomplishment by a user by tapping on the enclosure 102 of the wearable telemetry device 100. In a modification of the device of the invention, the wearable telemetry device 100 disclosed herein allows updating tasks. A user may tap the enclosure 102 of the wearable telemetry device 100 with a predetermined number of taps to confirm a task accomplishment on the user device 601 exemplarily illustrated in FIGS. 6-7. The device generates a sound via the speaker 418 to confirm that the event accomplishment was tracked after the device surface is tapped.

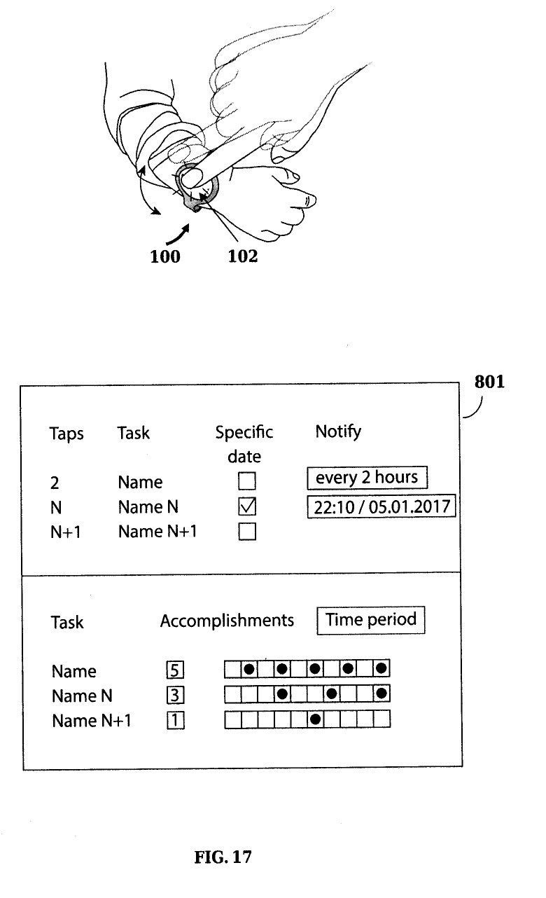

FIG. 18 exemplarily illustrates a user experience screenshot 801 provided by the monitoring application deployed on a user device 601 exemplarily illustrated in FIGS. 6-7, for setting up tasks and tracking accomplishment of the tasks. A user can use the wearable telemetry device 100 exemplarily illustrated in FIGS. 1A-1C and FIG. 17, to set up tasks. The user adds a task and assigns the number of taps on the enclosure 102 of the wearable telemetry device 100 exemplarily illustrated in FIG. 17, for the added task via the GUI 801 on the user device 601. The taps are needed to identify an accomplished task. The user can select a notification time from options, for example, periodical or a specific time and date via the GUI 801. The user can perform a task accomplishment confirmation by tapping on the enclosure 102 of the wearable telemetry device 100. To track the accomplished tasks, the GUI 801 displays a table with tasks, number of accomplishments, a timeline for the accomplishment of tasks for a selected period of time, etc., as exemplarily illustrated in FIG. 18. The GUI 801 displays the required information and controls. The monitoring application is the front-end application that provides the GUI 801 and is configured, for example, as an application for a smartphone, as an application for user device 601 such as a tablet computer, a personal computer, etc., as a web page, etc. The monitoring application enables simultaneous operation of several user devices 601 and collection of information on a state of health of several target objects, for example, children at any instance of time.

Thus, it has been shown that, even though some components of the wearable telemetry device of the invention are known per se, a combined use of all telemetric components of the system of the invention with comprehensive and interrelated data obtained from these components about vital signs, spatial orientation, and motions of the target object produce a novel and synergistic effect.

The foregoing examples have been provided merely for the purpose of explanation and are in no way to be construed as limiting of the method and the wearable telemetry device 100 disclosed herein. While the method and the wearable telemetry device 100 have been described with reference to various modifications, it is understood that the words, which have been used herein, are words of description and illustration, rather than words of limitation. Furthermore, although the method and the wearable telemetry device 100 have been described herein with reference to particular means, materials, and embodiments, the method and the wearable telemetry device 100 are not intended to be limited to the particulars disclosed herein; rather, the method and the wearable telemetry device 100 extend to all functionally equivalent structures, methods and uses, such as are within the scope of the appended claims. Those skilled in the art, having the benefit of the teachings of this specification, may affect numerous modifications thereto and changes may be made without departing from the scope and spirit of the method and the wearable telemetry device 100 disclosed herein in their aspects. For example, a target object is not necessarily a baby and may be an adult clinical patient, an in-house senior citizen suffering from Alzheimer's disease, or the like. The wearable telemetry device 100 may be attached to the body of a target object not necessarily with a bracelet but may be adhesively attached to the skin at any other part of the body.

What is claimed is:

1. A system for telemetrically monitoring a target object, which is a human being, the system comprising:
a wearable telemetry device for monitoring a target object comprising an enclosure that contains telemetry device components, a strap for detachably attaching and securing the wearable telemetry device to the target object in a position that provides contact of the enclosure with a target object surface and in a position in which the multiple telemetry device components detect target object parameters; and
at least one user device;
wherein the multiple telemetry device components produce sensor data and comprise: at least one processor, a self-contained power source, an audio input and an audio output connected to the at least one processor, an antenna connected to the at least one processor and to the user device via wireless communication units, an inertial block connected to the at least one processor, a PGG sensor block, a temperature sensor, a heart-rate output data determination and correlation block that is defined by a green LED and a photodiode, and an output SPO2 value determination and correlation block that is defined by a red LED, an infrared LED, and said photodiode, the heart-rate output data determination and correlation block and the output SPO2 value determination and correlation block each incorporating a function of checking reliability of the obtained sensor data and values, estimating an actual data by using a wavelet transformation, obtaining a corrected data, and calculating values by using the corrected data.

2. The system according to claim 2, wherein the human being is a baby, the enclosure is integrally connected to the strap, and wherein the strap has means for securing the wearable telemetry device on the wrist/arm of the baby.

3. The system according to claim 2, wherein the self-contained power source is a rechargeable battery and wherein the system is further comprising a base station which is powered from an external power source and has a wireless power charger which is inductively connected to the rechargeable battery.

4. The system according to claim 3, wherein the at least one processor comprises a plurality of modules comprising a sound control module connected to the audio input and an audio output, a spatial orientation module connected to the inertial block, a heart rate and SPO2 measurement control module connected to the PGG sensor block, a data transmitter/receiver module connected to the antenna via a wireless communication unit, a temperature measurement control module connected to the temperature sensor, an absolute height determination module connected to the spatial orientation module, a wavelet generation module, an ambient light filter, a DC filter, a data validation module, a vibration determination module, and an alerting module that generates alert notifications in multiple formats and transmits the generated alert notifications to the target object and/or the user devices via the wireless communication unit.

5. The system according to claim 4 wherein the inertial block comprises a 3-axis accelerometer, a 3-axis gyroscope, and a 3-axis magnetometer, and wherein the wireless communication units comprise a Bluetooth® transceiver module.

6. The system according to claim 4, further comprising a barometric sensor connected to the absolute height determination module.

7. The system according to claim 5, further comprising a barometric sensor connected to the absolute height determination module.

8. The system according to claim 3, further comprising means for initiating wireless transmission of the sensor data to the user device or the base station proximal to the wearable telemetry device via a wireless communication unit based on a deviation found in the sensor data, said means for initiating wireless transmission comprising a Bluetooth® transceiver module that is enabled in case of deviation of the data obtained from the sensors, a search device connectable to the wireless communication unit and/or the user device, and a terminal on the base station.

9. The system according to claim 1, wherein the user device comprises a device selected from the group consisting of a laptop, a smartphone, a tablet, and a computer, the user device comprising a monitoring application that has means for sending commands to the multiple telemetry device components via the wireless communication unit for marking a “safe” area around the target object and a user experience presentation screen.

10. The system according to claim 5, wherein the user device comprises a device selected from the group consisting of a laptop, a smartphone, a tablet, and a computer, the user device comprising a monitoring application that has means for sending commands to the multiple telemetry device components via the Bluetooth® transceiver module.

11. A method for telemetrically monitoring a target object, which is a human being, the method comprising the steps of:
providing a system comprising a wearable telemetry device for monitoring a target object comprising: an enclosure that contains telemetry device components, a device for attaching and securing the enclosure to the target object in contact with a target object surface in a position in which the multiple telemetry device components detect target object parameters and position, a wireless communication unit; and at least one user device, wherein the at least one user device is provided with means for detecting proximity of the target object and to dangerous spots, and with an alert module that generates alert notifications in multiple formats and transmits the generated alert notifications to the target object and/or the at least one user device;
attaching the wearable telemetry device to the target object by using the device for attaching and securing the enclosure to the target object;
telemetrically monitoring the target object parameters and the position relative to the dangerous spots by generating sensor data and for calculating data relating to health parameters of the target object comprising heart rate data, SpO2 value data, blood pressure, breathing rate, surface temperature of the target object, motion, and/position of the target object;
generating alert notifications in the alert notification module in multiple formats based on the calculated data;
constantly checking reliability of the calculated data; and
wirelessly transmitting the generated alert notifications to the target object and/or the user devices via a wireless communication unit to the user device;
wherein the step of constantly checking reliability of the calculated data comprises:
estimating the heart rate data obtained on the basis of green LED signals by using wavelet transformation, applying a signal pattern, and calculating a correlation data for the heart rate and a pattern;
estimating the SpO2 value data by using wavelet transformation,
determining AC by using signals obtained from red LED and infrared LED and obtaining a correlated data;
calculating the SpO2 value using the correlated data;
searching for the user device and/or base station; and
wirelessly transmitting the correlated data to the user device and/or to the base station.

12. The method according to claim 11, wherein the human being is a baby and the device for attaching and securing the enclosure to a target object in contact with a target object surface is strap attachable to the baby's wrist/arm in contact with the baby's skin.

13. The method according to claim 12, wherein the enclosure is integrally connected to the strap.

14. The method according to claim 13, wherein if the user device and/or the base station are not found, the wearable telemetry device waits for a predetermined time delay, assumes wireless communication with the user device and/or the base station, and continues to search for the user device and the base station.

15. The method according to claim 11, wherein the correlated data is transmitted directly between the wearable telemetry device and the user device via the wireless communication unit.

16. The method according to claim 11, wherein the correlated data is transmitted indirectly between the wearable telemetry device and the base station via a wireless communication unit and then by means of a home network segment to the user device.

17. The method according to claim 13, wherein the wireless communication unit is a Bluetooth® transceiver module and wherein the correlated data is transmitted directly between the wearable telemetry device and the user device via a Bluetooth® data transfer protocol.

18. The method according to claim 13, wherein the correlated data is transmitted indirectly between the wearable telemetry device and the base station by the Bluetooth® transceiver module and then by means of a home network segment to the user device.

19. The method according to claim 16, wherein in case of access of the user device or the home network segment to the Internet, the correlated data is stored in cloud resources and is available to other user devices.

20. The method according to claim 14, comprising the step of providing a user device with a monitoring application that comprises a user experience presentation screen.
